(19) United States
(12) Patent Application Publication
(10) Pub. No.: US 2026/0369409 A1
(43) Pub. Date: Nov. 19, 2026

(54) SYSTEM AND METHOD FOR 3D RECONSTRUCTION, ANALYSIS, AND INTERVENTION PLANNING OF BLOOD VESSELS USING ARTIFICIAL INTELLIGENCE

(71) Applicant: Define Medical Inc., San Francisco, CA (US)

(72) Inventor: Alejandro Rendon Sanchez, San Francisco, CA (US)

(73) Assignee: Define Medical Inc., San Francisco, CA (US)

(21) Appl. No.: 19/675,379

(22) Filed: May 12, 2026

Related U.S. Application Data
(60) Provisional application No. 63/804,909, filed on May 13, 2025.

(51) Int. Cl. G16H 50/20; A61B 34/10; G06T 3/18; G06T 7/33; G06T 7/62; G06T 17/20; G06V 10/26; G06V 10/764; G16H 20/40

(52) U.S. Cl. CPC G16H 50/20; A61B 34/10; G06T 3/18; G06T 7/337; G06T 7/62; G06T 17/20; G06V 10/26; G06V 10/764; G16H 20/40; A61B 2034/105; G06T 2207/20081; G06T 2207/20084; G06T 2207/30101; G06T 2210/41; G06V 2201/03

(57) ABSTRACT

In some embodiments, a computer-implemented method of facilitating treatment of vascular structures of a subject is provided. A computing system receives a plurality of medical images, and classifies the medical images of the plurality of medical images by views, angles, and points in a cardiac cycle. The computing system segments the medical images of the plurality of medical images to detect vascular structures. The computing system generates at least one three-dimensional model of the vascular structures using the classified and segmented plurality of medical images. The computing system conducts a mechanics analysis using the at least one three-dimensional model, and facilitates treatment of the vascular structures using a result of the mechanics analysis.

### CROSS-REFERENCE(S) TO RELATED APPLICATION(S)

This application claims the benefit of Provisional Application No. 63/804,909, filed May 13, 2025, the entire disclosure of which is hereby incorporated by reference herein for all purposes.

### BACKGROUND

Vascular structures can be visualized using various imaging modalities, including but not limited to x-ray angiography, computed tomography angiography (CT), and other techniques. Upon examination of the obtained imagery, diagnosis of various conditions may occur. Imaging may also be used to guide a catheter during treatment.

Existing techniques for x-ray angiography utilize static 2D imagery. Such techniques are unable to model the changes in structures over time (e.g., movement of vasculature during a complete cardiac cycle), and so various types of analysis that are relevant to diagnosis and treatment are not possible. What is desired are techniques that can build a 3D model from 2D imaging. What is also desired are techniques that, given a 3D model of the vasculature, can take motion of vasculature over time into account for diagnosis and determination of treatment strategies.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a computer-implemented method of facilitating treatment of vascular structures of a subject is provided. A computing system receives a plurality of medical images. The computing system classifies the medical images of the plurality of medical images by views, angles, and points in a cardiac cycle. The computing system segments the medical images of the plurality of medical images to detect vascular structures. The computing system generates at least one three-dimensional model of the vascular structures using the classified and segmented plurality of medical images. The computing system conducts a mechanics analysis using the at least one three-dimensional model. The computing system facilitates treatment of the vascular structures using a result of the mechanics analysis.

In some embodiments, a non-transitory computer-readable medium having computer-executable instructions stored thereon is provided. The instructions, in response to execution by one or more processors of a computing system, cause the computing system to perform actions for facilitating treatment of vascular structures of a subject. The actions comprise receiving, by the computing system, a plurality of medical images; classifying, by the computing system, the medical images of the plurality of medical images by views, angles, and points in a cardiac cycle; segmenting, by the computing system, the medical images of the plurality of medical images to detect vascular structures; generating, by the computing system, at least one three-dimensional model of the vascular structures using the classified and segmented plurality of medical images; conducting, by the computing system, a mechanics analysis using the at least one three-dimensional model; and facilitating, by the computing system, treatment of the vascular structures using a result of the mechanics analysis.

In some embodiments, a system comprising an imaging system and an image analysis system is provided. The imaging system is configured to generate medical images. The image analysis system comprises one or more processors and a non-transitory computer-readable medium having computer-executable instructions stored thereon. The instructions, in response to execution by the one or more processors, cause the image analysis system to perform actions comprising: receiving, by the computing system, a plurality of medical images generated by the imaging system; classifying, by the computing system, the medical images of the plurality of medical images by views, angles, and points in a cardiac cycle; segmenting, by the computing system, the medical images of the plurality of medical images to detect vascular structures; generating, by the computing system, at least one three-dimensional model of the vascular structures using the classified and segmented plurality of medical images; conducting, by the computing system, a mechanics analysis using the at least one three-dimensional model; and facilitating, by the computing system, treatment of the vascular structures using a result of the mechanics analysis.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic illustration of a system for collecting and analyzing medical images according to various aspects of the present disclosure.

FIG. 2 is a block diagram that illustrates aspects of a non-limiting example embodiment of an image analysis system according to various aspects of the present disclosure.

FIG. 3 is a flowchart that illustrates a non-limiting example embodiment of a method of processing medical images to guide treatment of vascular structures, according to various aspects of the present disclosure.

FIG. 4 is a flowchart that illustrates a non-limiting example embodiment of a subroutine for generating a three-dimensional model, according to various aspects of the present disclosure.

FIG. 5 is a flowchart that illustrates a non-limiting example embodiment of a subroutine for conducting a mechanics analysis, according to various aspects of the present disclosure.

FIG. 6 is a flowchart that illustrates a non-limiting example embodiment of processing three-dimensional CT-A data, according to various aspects of the present disclosure.

### DETAILED DESCRIPTION

The present disclosure relates to methods and systems for the reconstruction, analysis, and modeling of vascular structures (veins and arteries) using AI-based techniques. This technology integrates medical imaging with advanced computational methods to enhance diagnostics, surgical planning, and interventional procedures by providing real-time, precise reconstructions and characterizations of vascular anatomy. Some embodiments of the present disclosure provide a comprehensive solution for analyzing and reconstructing human anatomy, particularly blood vessels, from multiangle imaging data. It uses AI for view classification, geometric feature detection, 3D mesh creation, and deformation fitting to align models with anatomical templates. Additionally, some embodiments of the system quantify vascular characteristics, assess obstructions, and aid in intervention planning through resistance classification, tissue stiffness analysis, and optimal stent placement recommendations.

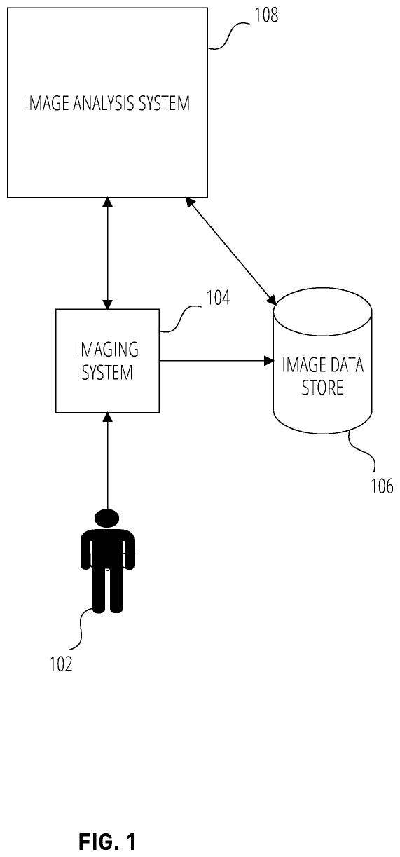

FIG. 1 is a schematic illustration of a system for collecting and analyzing medical images according to various aspects of the present disclosure. In the system, images of a subject 102 are collected by an imaging system 104. In some embodiments, the imaging system 104 is an x-ray angiography system, wherein an x-ray source and an image intensifier are positioned on a c-arm that is movable around the subject 102 to capture images from various angles without repositioning the subject 102. As known to those of ordinary skill in the art of x-ray angiography, the imaging system 104 may include additional hardware, including but not limited to control devices, storage devices, one or more catheters for delivery of contrast, etc. The use of x-ray angiography images should not be seen as limiting, as in some embodiments, other imaging modalities, including but not limited to CT scans and/or MRIs, may be used.

Once the images are generated by the imaging system 104, the images may be stored in an image data store 106. The image data store 106 may be separate from the imaging system 104 and the image analysis system 108, or may be integrated into either the imaging system 104 or the image analysis system 108. As used herein, “data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure. In some embodiments, the image data store 106 may be part of an electronic health record (EHR) system.

An image analysis system 108 receives the images that are generated by the imaging system 104. In some embodiments, the image analysis system 108 may receive the images directly from the imaging system 104 in real time or near-real time while they are collected. In some embodiments, the image analysis system 108 may retrieve the images from the image data store 106 after they are collected and stored. As discussed in further detail below, the image analysis system 108 is configured to build three-dimensional models of the vasculature depicted in the images, and to analyze the three-dimensional models in order to diagnose issues within the vasculature and suggest treatments to address the issues. In some embodiments, the image analysis system 108 may also be configured to determine optimal viewing angles from which the images should be collected, and may be configured to control the imaging system 104 to obtain the images from the optimal viewing angles.

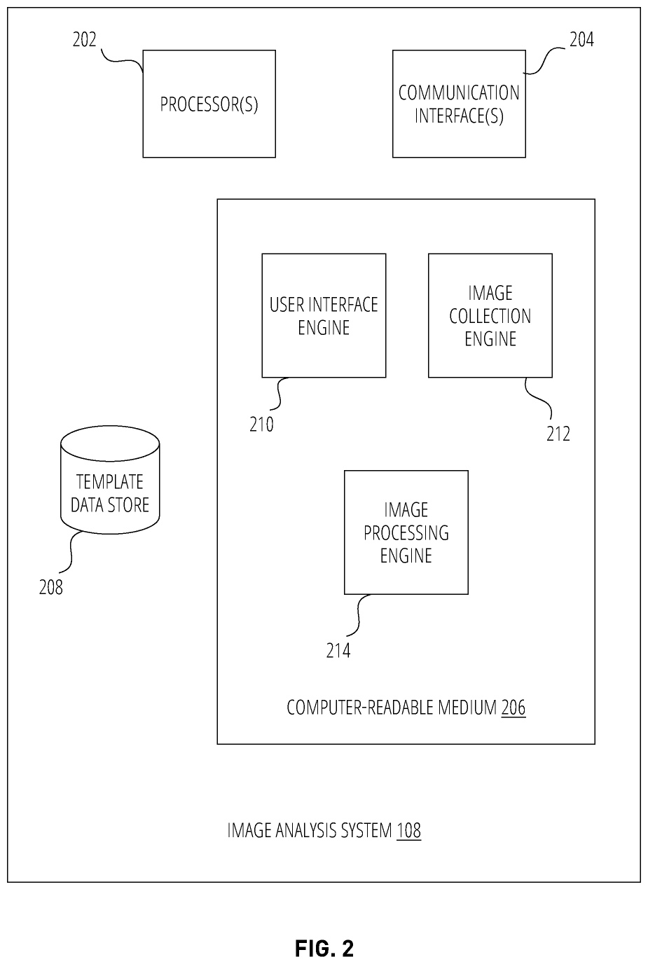

FIG. 2 is a block diagram that illustrates aspects of a non-limiting example embodiment of an image analysis system according to various aspects of the present disclosure. The illustrated image analysis system 108 may be implemented by any computing device or collection of computing devices, including but not limited to a desktop computing device, a laptop computing device, a mobile computing device, a server computing device, a computing device of a cloud computing system, and/or combinations thereof.

As shown, the image analysis system 108 includes one or more processors 202, one or more communication interfaces 204, a template data store 208, and a computer-readable medium 206.

In some embodiments, the processors 202 may include any suitable type of general-purpose computer processor. In some embodiments, the processors 202 may include one or more special-purpose computer processors or AI accelerators optimized for specific computing tasks, including but not limited to graphical processing units (GPUs), vision processing units (VPUs), and tensor processing units (TPUs).

In some embodiments, the communication interfaces 204 include one or more hardware and or software interfaces suitable for providing communication links between components. The communication interfaces 204 may support one or more wired communication technologies (including but not limited to Ethernet, FireWire, and USB), one or more wireless communication technologies (including but not limited to Wi-Fi, WiMAX, Bluetooth, 2G, 3G, 4G, 5G, and LTE), and/or combinations thereof.

As shown, the computer-readable medium 206 has stored thereon logic that, in response to execution by the one or more processors 202, cause the image analysis system 108 to provide a user interface engine 210, an image collection engine 212, and an image processing engine 214.

As used herein, “computer-readable medium” refers to a removable or nonremovable device that implements any technology capable of storing information in a volatile or non-volatile manner to be read by a processor of a computing device, including but not limited to: a hard drive; a flash memory; a solid state drive; random-access memory (RAM); read-only memory (ROM); a CD-ROM, a DVD, or other disk storage; a magnetic cassette; a magnetic tape; and a magnetic disk storage.

In some embodiments, the user interface engine 210 is configured to present images received by the image analysis system 108, to display generated three-dimensional models, and various other user interactions. For example, in some embodiments, a user may accept or reject images to be used to create the three-dimensional models, and/or may confirm or reject calculations performed by the image analysis system 108. In some embodiments, the user may select portions of the three-dimensional models or of the images for which metrics should be computed. In some embodiments, the user interface engine 210 may present the three-dimensional model alone, may present the three-dimensional model along with results of analysis (e.g., metrics, recommended treatment locations, etc.), or may present the three-dimensional model along with results of analysis and detailed curves.

In some embodiments, the image collection engine 212 is configured to receive images for processing. The image collection engine 212 may receive images directly from the imaging system 104, may retrieve previously stored images from the image data store 106, or combinations thereof. In some embodiments, the image collection engine 212 may be configured to instruct the imaging system 104 to collect images from a given angle or position with respect to the subject 102.

In some embodiments, the image processing engine 214 is configured to process the images collected by the image collection engine 212 in order to build three-dimensional models of the vasculature, to recommend treatment options, and/or to suggest optimal viewing angles for features of interest.

Further description of the configuration of each of these components is provided below.

As used herein, “engine” refers to logic embodied in hardware or software instructions, which can be written in one or more programming languages, including but not limited to C, C++, C#, COBOL, JAVA™, PHP, Perl, HTML, CSS, Javascript, VBScript, ASPX, Go, and Python. An engine may be compiled into executable programs or written in interpreted programming languages. Software engines may be callable from other engines or from themselves. Generally, the engines described herein refer to logical modules that can be merged with other engines, or can be divided into sub-engines. The engines can be implemented by logic stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device.

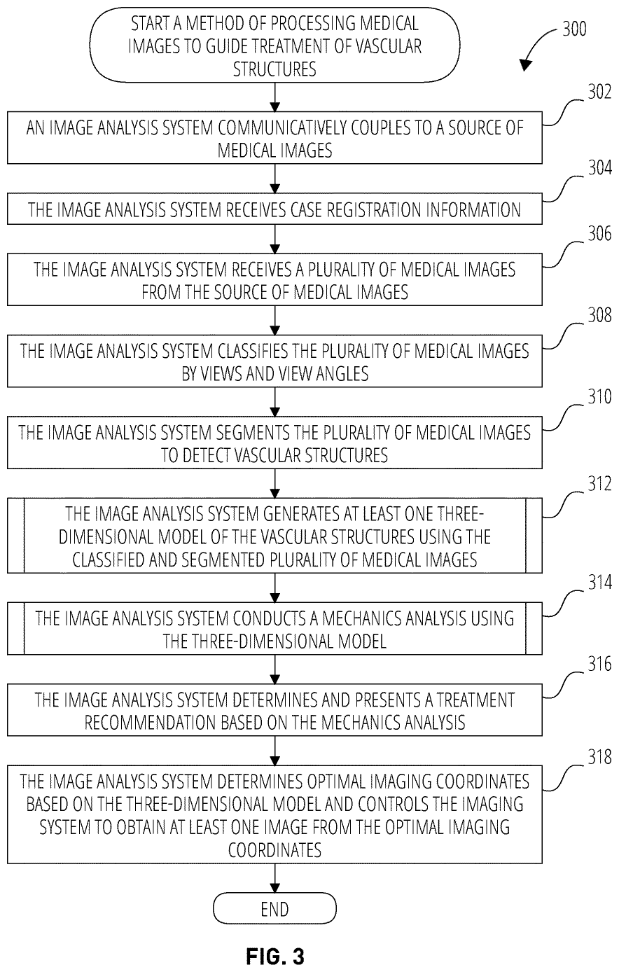

FIG. 3 is a flowchart that illustrates a non-limiting example embodiment of a method of processing medical images to guide treatment of vascular structures, according to various aspects of the present disclosure. In some embodiments, vascular structures to which the method 300 is applied are coronary arteries and the discussion below primarily discusses the analysis of coronary arteries, though in other embodiments, other vascular structures may be processed.

From a start block, the method 300 proceeds to block 302, where an image analysis system 108 communicatively couples to a source of medical images. In some embodiments, the image analysis system 108 may connect to the imaging system 104 or image data store 106 via a network or via a directly wired connection using the communication interfaces 204. The image analysis system 108 may present an indication that the connection was successfully established before proceeding.

At block 304, the image analysis system 108 receives case registration information. The case registration information may include an identifier of the subject 102, an electronic medical record, or other information. In some embodiments, the case registration information may be received directly from the imaging system 104 or image data store 106. For example, the connectivity from the image analysis system 108 to the imaging system 104 or image data store 106 may be through a Digital Imaging and Communication in Medicine (DICOM) node, the registration information may be received along with the images. In some embodiments, case registration information may not be received, or may be anonymized when presented by the user interface engine 210.

At block 306, the image analysis system receives a plurality of medical images from the source of medical images. In some embodiments, any standard imaging technique may be used. For example, for angiography images collected to image coronary arteries, a guide catheter may be inserted into the left or right coronary ostium. The imaging system 104 may be rotated and contrast may be injected to generate images and visualize coronary obstructions. Images may be captured for the left coronary artery (LCA) in any suitable view. As non-limiting examples, images may be captured in one or more of the following views:
1. CRA 35-40
2. CRA 25 LAO 25
3. CRA 30 RAO 30
4. CAU 30 LAO 30
5. CAU 20 RAO 20
6. Additional views including but not limited to CAU 30 collected as appropriate

The process may then be repeated to capture images of the right coronary artery (RCA). Images may be captured for the RCA in any suitable view. As non-limiting examples, images may be captured in one or more of the following views:
7. LAO 30
8. CRA 30 RAO 10
9. RAO 30
10. Additional views collected as appropriate

While this baseline angiogram is being collected, the live feed of the x-ray images may be presented by the user interface engine 210. In some embodiments, the x-ray planes and their coordinates are reported by the imaging system 104 and included with the plurality of images.

At block 308, the image analysis system 108 classifies the plurality of medical images by views and view angles. In some embodiments, the image analysis system 108 may use one or more classification models to automatically classify the views as depicting the LCA or RCA, and/or to classify the view angle. In some embodiments, the image analysis system 108 may use the x-ray planes and/or coordinates provided by the imaging system 104 as input to the classification model. Any suitable architecture may be used for the classification models, including but not limited to neural network models such as U-Net.

At block 310, the image analysis system 108 segments the plurality of medical images to detect vascular structures. In some embodiments, the medical images may be processed using a segmentation model that conducts a multi-class segmentation on the pixels of each image to determine which pixels depict vasculature and which pixels depict surrounding tissues. Any suitable segmentation model may be used, including but not limited to a convolutional neural network (CNN).

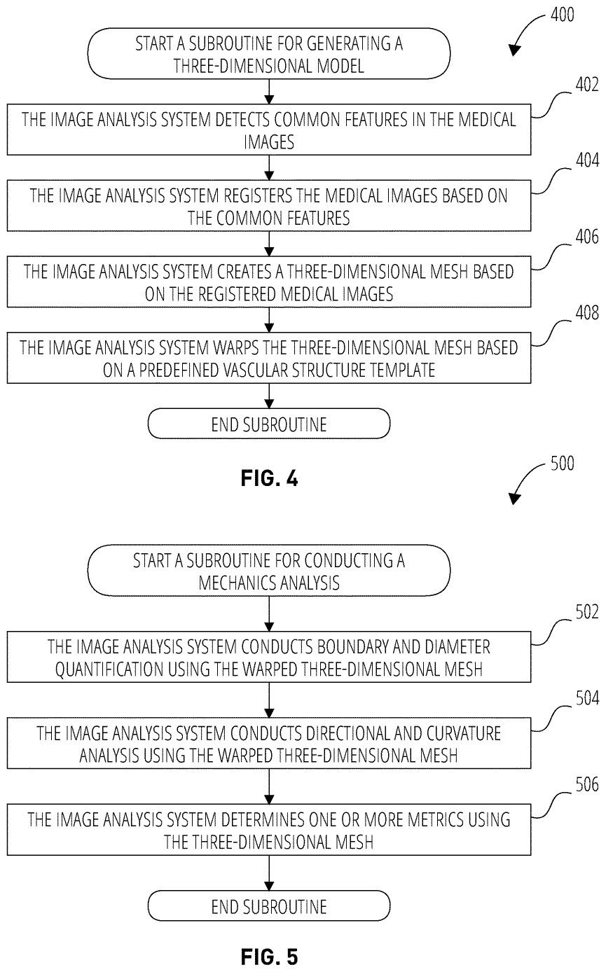

The method 300 then proceeds to subroutine block 312, where a subroutine is performed wherein the image analysis system 108 generates at least one three-dimensional model of the vascular structures using the classified and segmented plurality of medical images. Any suitable subroutine may be used to generate the three-dimensional model, including but not limited to the subroutine 400 illustrated in FIG. 4 and discussed in further detail below.

After subroutine block 312, the method 300 then proceeds to another subroutine block 314, where a subroutine is performed wherein the image analysis system 108 conducts a mechanics analysis using the three-dimensional model. Any suitable subroutine may be used to conduct the mechanics analysis, including but not limited to the subroutine 500 illustrated in FIG. 5 and discussed in further detail below. Upon completing subroutine block 312 and subroutine block 314, the image analysis system 108 has created a three-dimensional model of the vasculature of interest, and has identified points of interest within the model (e.g., points of restricted flow).

At block 316, the image analysis system 108 determines and presents a treatment recommendation based on the mechanics analysis. In some embodiments, the image analysis system 108 may recommend a stent with various characteristics based on the three-dimensional model (e.g., a length determined based on the detected blockage location, characteristics based on whether the blockage is at a straight section or near a bifurcation, an optimal balloon size, etc.). In some embodiments, the recommended stent may be an optimal stent chosen from a standardized catalog of available stents for implantation. In some embodiments, the image analysis system 108 may predict mechanical response of the vessel to specific interventional devices (including but not limited to intravascular lithotripsy (IVL), rotational atherectomy, orbital atherectomy, or standard balloon angioplasty) and may output device-specific procedural parameters to guide the interventionalist in real time.

In some embodiments, the image analysis system 108 utilizes a multi-modal AI-driven framework to optimize stent size and placement based on obstruction location, vessel geometry, and structural characteristics determined during subroutine block 312 and the subroutine block 314. To provide treatment recommendations, the AI-driven framework may be trained on a dataset of stent and balloon profiles, incorporating factors such as radial strength, flexibility, expansion dynamics, and compatibility with specific vessel morphologies. A deep reinforcement learning (DRL) algorithm, guided by supervised learning from historical procedural data, optimizes the stent selection process by evaluating prior clinical outcomes. Correlating subject-specific vessel characteristics (indicated by the three-dimensional model and the mechanics analysis) with a library of interventional devices, image analysis system 108 generates a treatment plan, suggesting the most suitable stent type, size, and positioning strategy to maximize patency while minimizing restenosis risk. In some embodiments, the user interface engine 210 presents the treatment plan to a user for implementation.

In some embodiments, determining a treatment recommendation based on the mechanics analysis may include predicting the mechanical response of the vasculature to specific interventional devices. The image analysis system 108 may operate on a set of stiffness-based decision thresholds to determine a recommended treatment. As non-limiting examples: In cases having high stiffness/low RWS with superficial calcium (consistent with calcified nodules or thick-cap calcified plaque), the image analysis system 108 may recommend rotational or orbital atherectomy for mechanical debulking, with specific burr size computed from the lumen geometry. In cases having high stiffness/low RWS with deep or circumferential calcium (consistent with concentric calcified sheets), the image analysis system 108 may recommend intravascular lithotripsy (IVL) for sonic fracturing, with pulse count and balloon sizing computed from the calcium arc and vessel diameter. In cases having moderate stiffness with mixed plaque, the image analysis system 108 may recommend high-pressure or super-high-pressure balloon predilatation, with inflation pressure computed from an FEA-derived compliance. In cases having low stiffness/high RWS (compliant, lipid-rich) the image analysis system 108 may recommend direct stenting or standard balloon predilatation. The image analysis system 108 may not merely a device category but specific hardware parameters: guidewire stiffness grade (e.g., standard vs. extra-support), balloon inflation pressure (atm), IVL pulse count and cycle parameters, and/or atherectomy burr sizing. These parameters may be derived from a library of device profiles that maps mechanical response characteristics of each device to the vessel's computed stiffness.

At block 318, the image analysis system 108 determines optimal imaging coordinates based on the three-dimensional model and controls the imaging system to obtain at least one image from the optimal imaging coordinates. The optimal imaging coordinates are the coordinates from which a clear, longitudinal view of the vascular segments of interest can be obtained, thus improving procedural outcomes. The optimal imaging coordinates may include a list of angle combinations that collectively provide the best view of the region of interest. In some embodiments, the image analysis system 108 utilizes real-time spatial registration of the heart of the subject 102 relative to the C-arm of the imaging system 104 to compute the optimal imaging angle and orientation. AI-driven algorithms may analyze vessel geometry, stenosis location, and C-arm kinematics, factoring in rotation, angulation, and translation dynamics to determine the best projection while minimizing foreshortening and vessel overlap. In some embodiments, the image analysis system 108 may use machine learning models trained on procedural data to predict optimal C-arm trajectories, dynamically adjusting positioning based on real-time fluoroscopic feedback and cardiac motion compensation. By solving for optimal kinematics, the optimal imaging coordinates generated by the image analysis system 108 enhance image clarity, reduce radiation exposure, and streamline procedural workflow.

In some embodiments, the method 300 may perform the treatment recommendation actions of block 316 without performing the imaging coordinate determination of block 318. In some embodiments, the method 300 may perform the imaging coordinate determination of block 318 without recommending a treatment action as described in block 316. In some embodiments, both the actions of block 316 and block 318 are performed in the method 300.

The method 300 then proceeds to an end block and terminates. Though illustrated as terminating, in some embodiments, the image analysis system 108 may collect results of the treatment (e.g., post-intervention imaging) to observe the vessel's mechanical response to the applied device, and differences between the observed response and the predicted response may be used to update the machine learning models used for the prediction. Over time, this feedback loop refines the system's ability to predict vessel response to specific devices, particularly improving accuracy for challenging lesion subtypes (e.g., calcified nodules, concentric calcification, mixed plaque, etc.). In some embodiments, the machine learning model may be trained on a database of post-IVL and post-atherectomy vessel responses, incorporating pre-intervention stiffness predictions, device parameters used, and post-intervention compliance changes. A deep reinforcement learning module may optimize the treatment recommendation by evaluating cumulative clinical outcomes (e.g., stent expansion, restenosis rates, acute gain, etc.) across the training database.

In the discussion above, it is assumed for ease of discussion that the plurality of medical images received at block 306 depict a matching point in a cardiac cycle, such that the size, shape, and location of the depicted vascular structures will be consistent. However, one of ordinary skill in the art will recognize that as the heart moves and blood circulates during the cardiac cycle, portions of the cardiac vasculature may be in different positions in relation to each other, may be under different amounts of pressure, may take on different shapes, and so on. Accordingly, it may be desired to obtain and process images from multiple points within a cardiac cycle in order to obtain a more comprehensive understanding of the physical characteristics of the vasculature.

Accordingly, in some embodiments of the present disclosure, the plurality of medical images includes images from different time points within a cardiac cycle (e.g., end-diastole, mid-systole, peak systole, early diastole, and/or other points of different granularity within the cardiac cycle). For example, when collecting images from a given view, the imaging system 104 may collect a plurality of images during one or more cardiac cycles, such that different points of the cardiac cycle are depicted from the given view. Such techniques that collect multiple images from a given view at different points of the cardiac cycle may be repeated for several different views.

In some embodiments, the medical images may be captured along with an electrocardiogram, and each image may be labeled with a particular point within the cardiac cycle with reference to the electrocardiogram. In some embodiments, the point in the cardiac cycle for a given image may be automatically determined using a machine learning technique similar to those discussed elsewhere herein for identifying a view or a vascular structure.

Once the plurality of medical images have been annotated with points in the cardiac cycle, images from different views that were captured at the same point during the cardiac cycle may be associated with each other, in order to be able to generate a consistent three-dimensional model of the state of the vasculature at that given point of the cardiac cycle. In some embodiments, such techniques may be used to build three-dimensional models for multiple different points during the cardiac cycle, so a three-dimensional representation of how the vasculature changes over time may be analyzed. The changes of the vasculature throughout the cardiac cycle may be additional inputs to the mechanics analysis conducted at subroutine block 314.

FIG. 4 is a flowchart that illustrates a non-limiting example embodiment of a subroutine for generating a three-dimensional model, according to various aspects of the present disclosure. The subroutine 400 is a non-limiting example of a subroutine suitable for use at subroutine block 312 of FIG. 3.

From a start block, the subroutine 400 proceeds to block 402, where the image analysis system 108 detects common features in the medical images. As discussed in FIG. 3, each image has already been segmented to indicate the pixels that depict vasculature versus surrounding tissue. At block 404, advance computational techniques may be used to identify features within the images (e.g., particular anatomical points in the vasculature). The approach may incorporate machine learning models, neural networks, pattern recognition algorithms, graph-based analysis, or other artificial intelligence methodologies to enhance accuracy and efficiency in object detection and classification. The image analysis system 108 may further employ novel techniques such as attention mechanisms, self-supervised learning, or alternative computational paradigms to improve image understanding and analysis.

In some embodiments, detecting common features may include performing analysis on each image to determine if the image quality is adequate. The image analysis system 108 may use techniques that use image contrast, clarity, radiologic density, and/or other aspects of the image to determine if the area for analysis is depicted in adequate detail for processing. If an image does not have adequate quality, the image analysis system 108 may skip further processing of the image and may instead use other images from the same view.

At block 404, the image analysis system 108 registers the medical images based on the common features. In some embodiments, the common features may include bifurcations, branch points, and/or other anatomical landmarks. In some embodiments, the image analysis system 108 may use a machine learning model (e.g., a classifier model) in order to determine a view for each medical image, and may register (e.g., align) images of matching views based on the locations of common features. The identity of the view defines the coordinates from which the image was captured. Accordingly, the two-dimensional images may be located within a three-dimensional volume by virtue of the identity of the view, and the images may then be registered to the three-dimensional volume by virtue of the location of matching common features in the separate images.

In some embodiments, in addition to registering the images in a three-dimensional volume, the image analysis system 108 may also register the images in time, such that images captured at or near the same time (or at or near the same point in a cardiac cycle) are aligned with each other. In some embodiments, computer vision techniques may be used to identify a point in a cardiac cycle to be used for time-based image registration. In some embodiments, an additional signal from an electrocardiogram (ECG) that is aligned to timestamps of the images may identify a point in a cardiac cycle for each image, such that images from the same point in the cardiac cycle may be registered.

At block 406, the image analysis system 108 creates a three-dimensional mesh based on the registered medical images. Since the registered medical images have been aligned to each other in the three-dimensional volume, and the pixels that identify vascular structures have been identified, known techniques can be used to determine the three-dimensional locations of the vasculature in order to create the three-dimensional mesh. Any suitable technique may be used, including creating a triangular or polygonal mesh from a point cloud indicated by projections of the classified pixels in the three-dimensional space.

At block 408, the image analysis system 108 warps the three-dimensional mesh based on a predefined vascular structure template. In some embodiments, an adaptive deformative adjustment framework may be used, wherein the reconstructed three-dimensional mesh undergoes dynamic alignment with a predefined anatomical template. Advanced deformation algorithms, potentially incorporating physics-informed neural networks, biomechanical modeling, or deep implicit functions, iteratively refine the mesh structure to ensure anatomical fidelity while accommodating patient-specific morphological variations. By warping the three-dimensional mesh to the predefined vascular structure template, the processing time may be reduced and the anatomical precision may be increased, while at the same time preserving anatomical accuracy and accounting for patient-specific variations. In some embodiments, predefined anatomical templates may be stored in the template data store 208, and the image analysis system 108 may determine a most-appropriate template to be used based on the views, by using a classifier model, or using any other suitable technique.

The subroutine 400 then proceeds to an end block and returns control to its caller, providing the warped three-dimensional mesh as a return value.

FIG. 5 is a flowchart that illustrates a non-limiting example embodiment of a subroutine for conducting a mechanics analysis, according to various aspects of the present disclosure. The subroutine 500 is a non-limiting example of a subroutine suitable for use at subroutine block 314 of FIG. 3.

From a start block, the subroutine 500 proceeds to block 502, where the image analysis system 108 conducts boundary and diameter quantification using the warped three-dimensional mesh. Points along the boundary of the vascular structure, as depicted in the three-dimensional model, are identified to calculate diameters and radii at various segments. The image analysis system 108 may detect geometric obstructions by comparing observed dimensions with standard vascular geometries.

At block 504, the image analysis system 108 conducts directional and curvature analysis using the warped three-dimensional mesh. The three-dimensional model's direction and curvature are characterized through an advanced computational framework that integrates inflow-outflow analysis with anatomical segmentation. A specialized algorithm analyzes the inflow at the coronary ostium and the corresponding outflow, enabling precise mapping of blood flow direction. This allows for an accurate representation of vascular dynamics in order to enable computing of blood flows. Additionally, a segmentation algorithm may be employed to identify bifurcations and morphological variations, ensuring that tortuous pathways and complex anatomical structures are accurately represented. By combining these elements, the system enhances procedural planning, optimizing navigation through patient-specific vascular geometries.

At block 506, the image analysis system 108 determines one or more metrics using the three-dimensional mesh. In some embodiments, the image analysis system 108 may assess flow dynamics within the vasculature, by computing the direction of flow, the velocity of flow, and various related metrics (e.g., percentage of flow, fractional flow reserve, etc.). In some embodiments, the image analysis system 108 may evaluate stiffness of the vasculature, by identifying normal versus abnormal areas and computing strain.

Various metrics useful for diagnosis and determination of treatment of vascular conditions may be computed using the three-dimensional mesh, including but not limited to anatomical resistance characterization (wherein obstructions are analyzed for their geometric constraints); fluid flow resistance characterization (wherein simulations of fluid dynamics are conducted to quantify resistance across different vascular sections), obstruction classification (wherein obstructions are classified based on resistance metrics, prioritizing areas requiring medical intervention), contrast washout calculation (wherein the image analysis system 108 calculates the washout rate of contrast agents in the vascular segments, thereby assessing perfusion and vascular integrity), contrast intensity indexing (wherein contrast intensity across vessel segments is indexed to support diagnostic evaluations), calcification identification (wherein calcifications are detected within vascular structures by using bone density as a reference, and wherein calcified plaques may be differentiated from surrounding tissues), and/or tissue stiffness calculation (wherein the stiffness of vascular tissues is quantified to assess elasticity in order to highlight areas of concern such as stenosis or plaque).

In some embodiments wherein multiple three-dimensional meshes that are labeled at different points within a cardiac cycle are available, metrics based on directly observed deformation of the vasculature at different points within the cardiac cycle may be determined. For example, the image analysis system 108 may compute Radial Wall Strain (RWS) at one or more interrogated lesion sites based on a maximum deformation of lumen diameter throughout the cardiac cycle, expressed as a percentage of the largest lumen diameter. The RWS computation may be performed segment-by-segment along the coronary tree, producing a spatial strain map that identifies regions of low deformation (indicative of high stiffness and calcification) and high deformation (indicative of compliant, lipid-rich plaque). This mechanical characterization is fundamentally different from radiodensity-based approaches: rather than inferring plaque composition from Hounsfield Units (HUs) in static CT, the system derives mechanical properties from observed behavior under physiological loading.

In some embodiments, to translate observed strain into quantitative stiffness values, the image analysis system 108 may perform Finite Element Analysis (FEA) on the vessel wall. Using the cardiac-phase-indexed cross-sectional geometries and measured intraluminal pressure differentials, the FEA module computes the stress-strain distribution across the vessel wall at each interrogated cross-section. The material model incorporates a Holzapfel-Gasser-Ogden constitutive framework adapted for arterial tissue, enabling characterization of both the circumferential and radial components of vessel wall mechanics.

The FEA output includes localized compliance values (expressed as diameter change per unit pressure), Young's modulus estimates for each lesion segment, and spatial maps of vessel wall stiffness. These quantities are used to classify each lesion into a stiffness tier that directly maps to the mechanical requirements of available interventional devices.

In some embodiments, the mechanics analysis may be augmented with data obtained via different modalities. In embodiments wherein invasive pressure wire data is available (as is common during diagnostic catheterization), the image analysis system 108 may use the measured pressure gradient across a stenosis to calibrate and verify the non-invasive stiffness calculations derived from the strain analysis described above. The invasive pressure-diameter relationship provides a ground-truth anchor for the FEA-derived compliance values, reducing model uncertainty and improving the accuracy of treatment recommendations.

In embodiments where CT angiography (CTA) data is also available, the image analysis system 108 may be configured to perform Hounsfield Unit-based compliance mapping: the radiodensity profile of a lesion from CTA is correlated with the strain-derived stiffness described above, creating a multi-source confidence score for the mechanical characterization. This multisource integration—combining CTA morphology, angiographic dynamics, and invasive hemodynamics—constitutes a novel Anatomy+Physiology+Mechanics (A+P+M) framework that provides additional benefits over existing systems.

The subroutine 500 then proceeds to an end block and returns control to its caller, with the various metrics and other analysis output provided as return value(s).

The method 300 and subroutines 400, 500 described above were discussed in the context of medical images such as two-dimensional X-ray angiograph images. However, in some embodiments, three-dimensional CT volumes may be received instead of two-dimensional images, and the segmentation of block 310 and the construction of the three-dimensional model in subroutine block 312 may be replaced with processing that uses the three-dimensional CT volumes to directly construct a three-dimensional model that includes at least the surface mesh and is usable for the mechanics analysis of subroutine block 314.

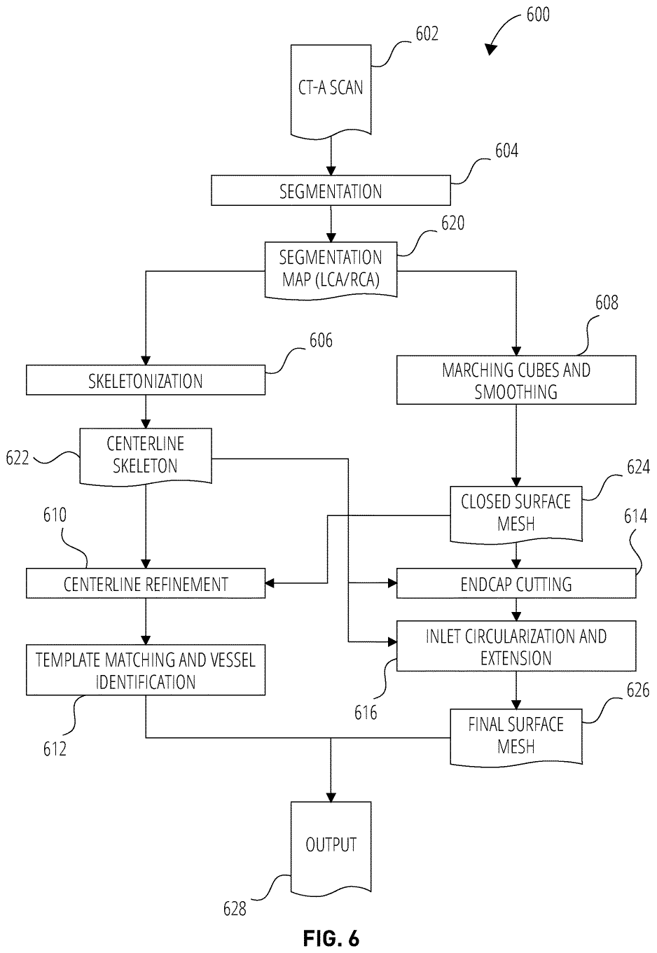

FIG. 6 is a flowchart that illustrates a non-limiting example embodiment of processing three-dimensional CT-A data, according to various aspects of the present disclosure. Compared to a conventional segmentation task, the proposed coronary artery (CA) extraction workflow supports the comprehensive anatomical and functional analyses described above. In addition to generating voxel-level segmentation outputs, the workflow enables a segmentation map, a surface mesh, vessel identification, and length vs radius measurements. In some embodiments, the segmentation map may identify voxels belonging to the left coronary artery (LCA) and right coronary artery (RCA), providing an anatomically consistent voxel-level delineation of the coronary tree. In some embodiments, the surface mesh is a smoothed three-dimensional surface representation of the coronary vessel trees, with inlets and outlets processed and prepared for subsequent computational fluid dynamics (CFD) simulations. In some embodiments, vessel identification is achieved when extracted vessel centerlines are compared against a set of pre-annotated anatomical templates to further subdivide the coronary trees into their constituent vessels. In some embodiments, length versus radius measurements are used such that vessel radius distributions along the vessel centerlines are computed using the extracted surface mesh and centerline information, enabling quantitative analysis of vessel morphology.

As shown in the flowchart 600, once a CT-A scan 602 is received, segmentation is performed at block 604 to create a segmentation map 620. In some embodiments, a 3D U-Net-based convolutional neural network may be used to perform volumetric segmentation of the left and right coronary artery trees from contrast-enhanced CT-A scans. The U-Net architecture was selected for its encoder-decoder design with skip connections, which enables effective multi-scale feature extraction while preserving fine-grained spatial details critical for delineating small-diameter coronary vessels. In some embodiments, the U-Net may process volumetric image patches with size 256×256×256 as input and produces voxel-level predictions distinguishing the left coronary artery (LCA) and right coronary artery (RCA), allowing anatomically consistent separation of the coronary trees.

For model training, a combination of Dice loss and total variation loss may be used employed. Dice loss was used to optimize overlap with the provided ground truth annotations, directly supporting accurate voxel-level segmentation of the coronary arteries. Total variation loss may be incorporated as a regularization term to encourage spatial smoothness of the predicted segmentation, thereby reducing discontinuities along the vessel body and suppressing isolated false-positive predictions.

The performance of a non-limiting example embodiment of this segmentation model was evaluated in comparison with existing coronary artery segmentation approaches, including TotalSegmentator and the coronary artery segmentation model described by (Zeng et al., 2023). Quantitative evaluation results are summarized in the table below.

| | Total | Zeng et al., | | | |
|---|---|---|---|---|---|
| | Segmentator | 2023 | Segmentation Unet | | |
| | Binary task | Binary task | Binary task | RCA | LCA |
| Dice score | 0.45 | 0.83 | 0.82 | 0.81 | 0.80 |

The performance of the proposed segmentation U-Net on the binary segmentation task falls within the reported error margins of the best-performing ensemble model described by (Zeng et al., 2023). The slightly reduced performance observed in the multi-label segmentation task is primarily attributable to misclassification between the left and right coronary artery segments, particularly in regions adjacent to the cardiac septum.

Once the segmentation map 620 is obtained, the volumetric segmentation map 620 is converted into a closed surface mesh 624, using techniques such as marching cubes and smoothing in block 608. Converting the volumetric segmentation mask into a surface mesh representation enables the coronary arterial tree to be modeled as a high-quality triangular mesh suitable for the computational fluid dynamics (CFD) simulations described above. This transformation provides an explicit geometric description of the vessel lumen, which is useful for accurate numerical modeling of blood flow, pressure gradients, and wall shear stress. Compared to voxel-based representations, surface meshes offer smoother boundaries, reduced numerical diffusion, and improved control over boundary conditions at vessel inlets and outlets. As a result, the mesh-based representation serves as a useful intermediate step for physiologically meaningful CFD analysis, supporting the downstream applications such as hemodynamic assessment, disease characterization, and treatment planning discussed above.

To create the closed surface mesh 624 at block 608, the marching cubes algorithm is first applied to the largest connected component of each label in the segmentation mask to generate a closed surface representation of the arterial tree. To mitigate artifacts introduced by the relatively coarse spatial resolution of CT voxels compared with the expected range of coronary vessel radii, the resulting surface mesh may subsequently be refined using a combination of Taubin smoothing and simple neighborhood-averaging smoothing operations.

Following surface reconstruction and smoothing, the flowchart 600 proceeds to block 614 and block 616 to perform mesh-processing steps to further prepare the closed surface mesh 624 for CFD simulation and thereby create a final surface mesh 626. At block 614, end caps are removed. The closed surface mesh 624 is truncated in the vicinity of the coronary ostium and at the distal ends of each vessel branch to create a single inlet and multiple outlets. Each cut is defined by a plane oriented perpendicular to the local vessel centerline and positioned to eliminate artificial vessel narrowing caused by proximity to the segmentation boundary. At block 616, each inlet is processed individually to meet CFD boundary condition requirements. In cases where the distance between the original inlet location and the first bifurcation is less than 10 mm, the inlet is artificially extended to ensure sufficient flow development length. In addition, the inlet cross-section is circularized to provide a well-defined and numerically stable boundary for CFD simulations.

In addition to generating the final surface mesh 626, the segmentation map 620 may also be used to extract a vessel centerline of the coronary artery tree, which may then be used for various purposes. Extracting a vessel centerline from a volumetric segmentation of the coronary artery tree is a useful step for enabling anatomically meaningful analysis and downstream processing. The centerline provides a compact, topology-preserving representation of the arterial tree that captures vessel connectivity and branching structure while reducing the complexity of the full volumetric or surface-based representation. In addition, centerlines serve as a geometric reference for measuring clinically relevant quantities such as vessel length and radius profiles along the vessel path, and they facilitate consistent labeling and identification of individual coronary branches.

At block 606, the volumetric segmentation map 620 is skeletonized to generate a set of three-dimensional points located near the geometric center of the segmented vessels, providing an initial approximation of the centerline skeleton 622. In some embodiments, the centerline skeleton 622 may be provided to block 614 and/or block 616 to assist with those actions with respect to creating the final surface mesh 626. Likewise, the closed surface mesh 624 may be provided to block 610 to assist with refinement of the centerline.

Various actions may then be performed at block 610 to refine the centerline skeleton 622. In some embodiments, a graph structure is constructed over the skeleton points by identifying connectivity relationships in order to establish point connectivity. In some embodiments, endpoints and bifurcation points are detected based on the number of neighboring connected points, allowing reconstruction of the vessel topology and branching structure. In some embodiments, very short branches introduced during the skeletonization process at block 606 may be pruned from the graph to prevent surface irregularities from being misinterpreted as true vessel branches. In some embodiments, the centerline skeleton 622 may be refined using Laplacian smoothing to reduce discretization noise introduced during skeletonization. To preserve vessel length and topology, endpoints and bifurcation points may be held fixed during the smoothing process, in order to minimize undesired centerline shortening.

Once the centerline skeleton 622 has been refined, template matching and vessel identification may be performed at block 618. Decomposing the coronary arterial trees into their constituent vessels, followed by cross-identification with anatomically corresponding structures, assists with clinical usability, as it helps enable the association of the extracted geometric and hemodynamic properties with specific, anatomically defined vessels, thereby supporting meaningful interpretation and downstream analysis.

The vessel identification procedure is based on a template-fitting approach, in which each centerline segment extracted from the segmented vessel tree is assigned to a corresponding anatomical vessel defined in an annotated template. To support robust vessel identification, a set of reference templates was manually annotated for both the LCA and RCA trees (see Section Data Sources). The process of establishing correspondence between centerline segments and template vessels includes initial registration, pruning of the template model, final registration, and a sanity check. For initial registration, a sequence of rigid and non-rigid registration steps is applied to the centerline graph to achieve an initial alignment with the template model. To prune the template model, the mean average distance between each centerline component and each template vessel is computed, and template vessels for which no corresponding centerline segment is identified, indicated by consistently large mean average distances, are removed from the template model. For final registration, the registration process is repeated using the pruned template model to refine the alignment. Based on the updated distance measurements, correspondence between individual centerline segments and anatomical vessels is established. An additional validation step is applied as a sanity check to ensure that the connectivity between the identified vessel segments is anatomically consistent with known coronary artery topology. This procedure is repeated for each available template model, and the vessel identification result is selected from the template that achieves the highest overall matching score, as determined by the mean average distance between the optimally matched centerline segments and template vessels.

Once the final surface mesh 626 and vessel identification has been completed, the flowchart 600 produces an output 628. The output 628 includes the final surface mesh 626, and may include indications of one or more vessel components such as the center line points, radii, and/or length. In some embodiments, the output 628 may also include surface colormaps. This output 628 may then be processed as discussed in FIG. 3 in order to suggest and guide treatment options.

While illustrative embodiments have been illustrated and described, it will be appreciated that various changes can be made therein without departing from the spirit and scope of the invention.

### EXAMPLES

The following paragraphs provide a set of numbered, non-limiting example embodiments of the subject matter disclosed herein.

Example 1: A computer-implemented method of facilitating treatment of vascular structures of a subject, the method comprising: receiving, by a computing system, a plurality of medical images; classifying, by the computing system, the medical images of the plurality of medical images by views, angles, and points in a cardiac cycle; segmenting, by the computing system, the medical images of the plurality of medical images to detect vascular structures; generating, by the computing system, at least one three-dimensional model of the vascular structures using the classified and segmented plurality of medical images; conducting, by the computing system, a mechanics analysis using the at least one three-dimensional model; and facilitating, by the computing system, treatment of the vascular structures using a result of the mechanics analysis.

Example 2: The computer-implemented method of example 1, wherein generating the at least one three-dimensional model of the vascular structures using the classified and segmented plurality of medical images includes: generating a plurality of three-dimensional models of the vascular structures, wherein each three-dimensional model is at a different point in the cardiac cycle.

Example 3: The computer-implemented method of example 2, wherein conducting the mechanics analysis using the at least one three-dimensional model includes: conducting, by the computing system, the mechanics analysis based on differences within the plurality of three-dimensional models.

Example 4: The computer-implemented method of any one of examples 1-3, wherein facilitating treatment of the vascular structures using the result of the mechanics analysis includes recommending a treatment; wherein recommending the treatment includes recommending at least one of a procedure type, a location, a stent length, a balloon size, a guidewire stiffness grade, a balloon inflation pressure, IVL pulse count and cycle parameters, or atherectomy burr sizing.

Example 5: The computer-implemented method of any one of examples 1-4, wherein facilitating treatment of the vascular structures using the results of the mechanics analysis includes determining optimal imaging coordinates, and wherein the method further comprises: transmitting commands to an imaging system to cause the imaging system to collect images from the optimal imaging coordinates.

Example 6: The computer-implemented method of any one of examples 1-5, wherein generating the at least one three-dimensional model of the vascular structures using the classified and segmented plurality of medical images includes: detecting common features in the medical images; registering the medical images in a three-dimensional volume based on the common features; and creating a three-dimensional mesh based on the registered medical images.

Example 7: The computer-implemented method of example 6, further comprising warping the three-dimensional mesh based on a predefined vascular structure template.

Example 8: The computer-implemented method of any one of examples 1-7, wherein conducting the mechanics analysis using the three-dimensional model includes: conducting boundary and diameter quantification using the three-dimensional model; conducting directional and curvature analysis using the three-dimensional model; and determining one or more metrics using the three-dimensional model.

Example 9: The computer-implemented method of example 8, wherein the one or more metrics include at least one of: an anatomical resistance characterization; a fluid flow resistance characterization; an obstruction classification; a contrast washout calculation; contrast intensity indexing; a calcification identification; or a tissue stiffness calculation.

Example 10: The computer-implemented method of any one of examples 1-9, wherein receiving the plurality of medical images includes at least one of: receiving the plurality of medical images from an imaging system in real time from an x-ray angiography system, a CT scanning system, or an MRI system; or retrieving the plurality of medical images from an image data store.

Example 11: A non-transitory computer-readable medium having computer-executable instructions stored thereon that, in response to execution by one or more processors of a computing system, cause the computing system to perform actions for facilitating treatment of vascular structures of a subject, the actions comprising: receiving, by the computing system, a plurality of medical images; classifying, by the computing system, the medical images of the plurality of medical images by views, angles, and points in a cardiac cycle; segmenting, by the computing system, the medical images of the plurality of medical images to detect vascular structures; generating, by the computing system, at least one three-dimensional model of the vascular structures using the classified and segmented plurality of medical images; conducting, by the computing system, a mechanics analysis using the at least one three-dimensional model; and facilitating, by the computing system, treatment of the vascular structures using a result of the mechanics analysis.

Example 12: The non-transitory computer-readable medium of example 11, wherein generating the at least one three-dimensional model of the vascular structures using the classified and segmented plurality of medical images includes: generating a plurality of three-dimensional models of the vascular structures, wherein each three-dimensional model is at a different point in the cardiac cycle.

Example 13: The non-transitory computer-readable medium of example 12, wherein conducting the mechanics analysis using the at least one three-dimensional model includes: conducting, by the computing system, the mechanics analysis based on differences within the plurality of three-dimensional models.

Example 14: The non-transitory computer-readable medium of any one of examples 11-13, wherein facilitating treatment of the vascular structures using the result of the mechanics analysis includes recommending a treatment; wherein recommending the treatment includes recommending at least one of a procedure type, a location, a stent length, a balloon size, a guidewire stiffness grade, a balloon inflation pressure, IVL pulse count and cycle parameters, or atherectomy burr sizing.

Example 15: The non-transitory computer-readable medium of any one of examples 11-14, wherein facilitating treatment of the vascular structures using the results of the mechanics analysis includes determining optimal imaging coordinates, and wherein the actions further comprise: transmitting commands to an imaging system to cause the imaging system to collect images from the optimal imaging coordinates.

Example 16: The non-transitory computer-readable medium of any one of examples 11-15, wherein generating the at least one three-dimensional model of the vascular structures using the classified and segmented plurality of medical images includes: detecting common features in the medical images; registering the medical images in a three-dimensional volume based on the common features; and creating a three-dimensional mesh based on the registered medical images.

Example 17: The non-transitory computer-readable medium of example 16, wherein the actions further comprise warping the three-dimensional mesh based on a predefined vascular structure template.

Example 18: The non-transitory computer-readable medium of any one of examples 11-17, wherein conducting the mechanics analysis using the three-dimensional model includes: conducting boundary and diameter quantification using the three-dimensional model; conducting directional and curvature analysis using the three-dimensional model; and determining one or more metrics using the three-dimensional model.

Example 19: The non-transitory computer-readable medium of example 18, wherein the one or more metrics include at least one of: an anatomical resistance characterization; a fluid flow resistance characterization; an obstruction classification; a contrast washout calculation; contrast intensity indexing; a calcification identification; or a tissue stiffness calculation.

Example 20: A system, comprising: an imaging system configured to generate medical images; and an image analysis system comprising one or more processors and a non-transitory computer-readable medium having computer-executable instructions stored thereon that, in response to execution by the one or more processors, cause the image analysis system to perform actions comprising: receiving, by the computing system, a plurality of medical images generated by the imaging system; classifying, by the computing system, the medical images of the plurality of medical images by views, angles, and points in a cardiac cycle; segmenting, by the computing system, the medical images of the plurality of medical images to detect vascular structures; generating, by the computing system, at least one three-dimensional model of the vascular structures using the classified and segmented plurality of medical images; conducting, by the computing system, a mechanics analysis using the at least one three-dimensional model; and facilitating, by the computing system, treatment of the vascular structures using a result of the mechanics analysis.

Example 21: The system of example 20, wherein the imaging system includes at least one of an x-ray angiography system, a CT scanning system, or an MRI system.

Example 22: A system for intraprocedural guidance of coronary interventions, the system comprising: one or more processors; and a non-transitory computer-readable medium storing instructions that, when executed by the one or more processors, cause the system to: (a) receive a time-resolved angiographic sequence comprising a plurality of image frames of a coronary vascular structure captured across a plurality of phases of a cardiac cycle; (b) reconstruct, from the time-resolved angiographic sequence, a plurality of phase-indexed lumen geometries, each phase-indexed lumen geometry corresponding to a respective phase of the cardiac cycle; (c) compute, for each of one or more interrogated lesion segments within the coronary vascular structure, a Radial Wall Strain (RWS) value based on deformation of a lumen diameter across the plurality of phase-indexed lumen geometries; (d) derive, from the RWS value and a computational model of vessel wall mechanics, a localized vessel stiffness metric for each interrogated lesion segment; (e) classify, based on the localized vessel stiffness metric, each interrogated lesion segment into a stiffness tier selected from a plurality of predefined stiffness tiers; and (f) generate, based on the stiffness tier, a treatment recommendation comprising an automated selection between at least two of: (i) mechanical debulking by rotational or orbital atherectomy, (ii) sonic fracturing by intravascular lithotripsy (IVL), or (iii) balloon-mediated vessel preparation, wherein the treatment recommendation is output to a user interface for intraprocedural guidance.

Example 23. The system of example 22, wherein step (a) further comprises receiving at least one of: a computed tomography angiography (CTA) dataset of the coronary vascular structure, wherein the CTA dataset provides Hounsfield Unit (HU) values for plaque characterization; or an invasive pressure measurement obtained via a pressure wire positioned within the coronary vascular structure, and wherein the instructions further cause the system to integrate the CTA dataset, the invasive pressure measurement, or both with the time-resolved angiographic sequence to compute the localized vessel stiffness metric using a multisource fusion algorithm.

Example 24. The system of any one of examples 22-23, wherein the treatment recommendation further comprises one or more hardware parameters selected from: a guidewire stiffness grade; a balloon inflation pressure expressed in atmospheres; an atherectomy burr size computed from the lumen geometry; an IVL balloon sizing and pulse count; or a combination thereof, wherein the one or more hardware parameters are derived from a device profile library that maps mechanical response characteristics of interventional devices to the localized vessel stiffness metric.

Example 25. The system of any one of examples 22-24, wherein the instructions further cause the system to: receive post-intervention imaging data of the coronary vascular structure after application of a selected interventional device; compute a post-intervention vessel compliance from the post-intervention imaging data; compare the post-intervention vessel compliance with a pre-intervention stiffness prediction to generate a prediction residual; and update a machine learning model used in step (d) based on the prediction residual, wherein the machine learning model is trained on a database of post-intravascular-lithotripsy and post-atherectomy vessel responses to improve accuracy of the localized vessel stiffness metric for subsequent patients.

Example 26. The system of any one of examples 22-25, wherein the instructions further cause the system to: receive invasive pressure wire data comprising a pressure gradient measured across a stenosis within the coronary vascular structure; compute a pressure-diameter relationship from the invasive pressure wire data and a corresponding phase-indexed lumen geometry; and calibrate the localized vessel stiffness metric derived in step (d) using the pressure-diameter relationship as a ground-truth reference, thereby performing cross-modality calibration between the invasive pressure measurement and the non-invasive stiffness calculation derived from the time-resolved angiographic sequence.

Example 27. The system of any one of examples 22-26, wherein deriving the localized vessel stiffness metric in step (d) comprises: performing Finite Element Analysis (FEA) on a plurality of phase-indexed cross-sectional geometries of the interrogated lesion segment, wherein the FEA employs a constitutive material model for arterial tissue; computing, from the FEA, a stress-strain distribution across the vessel wall at each interrogated cross-section; and deriving the localized vessel stiffness metric as a function of the computed stress-strain distribution, wherein the localized vessel stiffness metric comprises at least one of: a localized compliance value, a Young's modulus estimate, or a spatial map of vessel wall stiffness.

Example 28. A system for intraprocedural guidance of coronary interventions, the system comprising one or more processors; and a non-transitory computer-readable medium storing instructions that, when executed by the one or more processors, cause the system to: (a) receive time-resolved imaging data of a coronary vascular structure captured across a plurality of phases of a cardiac cycle, wherein the time-resolved imaging data is obtained by means for acquiring temporally-resolved coronary images including at least one of: x-ray angiography, intravascular ultrasound, optical coherence tomography, computed tomography with cardiac gating, or magnetic resonance angiography; (b) reconstruct, from the time-resolved imaging data, a plurality of phase-indexed vessel geometries, each phase-indexed vessel geometry representing a spatial configuration of the coronary vascular structure at a respective phase of the cardiac cycle; (c) compute, for each of one or more interrogated lesion segments, a deformation metric quantifying dimensional variation of the vessel across the plurality of phase-indexed vessel geometries, the deformation metric being derived by means for measuring cyclic vessel deformation including at least one of: radial wall strain, luminal diameter variation, cross-sectional area change, circumferential strain, or any functionally equivalent measure of vessel wall displacement across the cardiac cycle; (d) derive, from the deformation metric, a localized mechanical property characterizing the stiffness, compliance, or elasticity of the vessel wall at each interrogated lesion segment, the localized mechanical property being computed by means for biomechanical characterization including at least one of: Finite Element Analysis, boundary element methods, mesh-free computational methods, analytical pressure-diameter models, physics-informed neural networks, or any functionally equivalent computational technique for determining vessel wall mechanical properties from observed deformation; (e) determine, based on the localized mechanical property, a treatment strategy for each interrogated lesion segment, the treatment strategy being generated by means for mapping mechanical properties to interventional actions, the treatment strategy comprising a selection among a plurality of calcium modification or vessel preparation modalities; and (f) output the treatment strategy to a user interface or to a connected interventional system for intraprocedural guidance.

Example 29. The system of example 28, wherein step (a) further comprises receiving imaging data from a plurality of imaging modalities, the plurality of imaging modalities including at least two of: x-ray angiography, computed tomography angiography, intravascular ultrasound, optical coherence tomography, or invasive pressure measurement, and wherein the instructions further cause the system to fuse the imaging data from the plurality of imaging modalities using a multisource integration module to compute the localized mechanical property with higher confidence than achievable from any single modality alone.

Example 30. The system of any one of examples 28-29, wherein the treatment strategy output in step (f) further comprises one or more device-specific procedural parameters determined by means for computing hardware specifications from vessel mechanics, the one or more device-specific procedural parameters including at least one of: a guidewire support level, a balloon inflation pressure, a calcium modification device sizing, a pulse or cycle count for a lithotripsy device, a rotational speed or burr dimension for an atherectomy device, or any functionally equivalent hardware parameter derived from the localized mechanical property.

Example 31. The system of any one of examples 28-30, wherein the instructions further cause the system to: receive post-intervention imaging data; compute a post-intervention mechanical property of the vessel; and update, using a machine learning model, the means for biomechanical characterization based on a comparison between a pre-intervention prediction and the post-intervention mechanical property, thereby implementing a closed-loop refinement of stiffness estimation accuracy across a population of patients.

Example 32. The system of any one of examples 28-31, wherein the instructions further cause the system to: receive invasive hemodynamic data comprising at least one of a pressure gradient, a flow velocity measurement, or a pullback pressure profile obtained within the coronary vascular structure; and calibrate the localized mechanical property derived in step (d) using the invasive hemodynamic data as a reference standard, thereby performing cross-modality verification between invasive measurements and non-invasive biomechanical computations.

Example 33. The system of any one of examples 28-32, wherein the deformation metric in step (c) is used as a surrogate for plaque calcification burden, wherein regions of the coronary vascular structure exhibiting deformation metric values below a predefined threshold are classified as having elevated calcium content, and wherein the localized mechanical property derived in step (d) is correlated with radiodensity data from computed tomography to generate a composite calcium confidence score, the composite calcium confidence score being used by the means for mapping mechanical properties to interventional actions to prioritize calcium modification modalities in the treatment strategy.

The embodiments of the invention in which an exclusive property or privilege is claimed are defined as follows:

1. A computer-implemented method of facilitating treatment of vascular structures of a subject, the method comprising:
receiving, by a computing system, a plurality of medical images;
classifying, by the computing system, the medical images of the plurality of medical images by views, angles, and points in a cardiac cycle;
segmenting, by the computing system, the medical images of the plurality of medical images to detect vascular structures;
generating, by the computing system, at least one three-dimensional model of the vascular structures using the classified and segmented plurality of medical images;
conducting, by the computing system, a mechanics analysis using the at least one three-dimensional model; and
facilitating, by the computing system, treatment of the vascular structures using a result of the mechanics analysis.

2. The computer-implemented method of claim 1, wherein generating the at least one three-dimensional model of the vascular structures using the classified and segmented plurality of medical images includes:
generating a plurality of three-dimensional models of the vascular structures, wherein each three-dimensional model is at a different point in the cardiac cycle.

3. The computer-implemented method of claim 2, wherein conducting the mechanics analysis using the at least one three-dimensional model includes:
conducting, by the computing system, the mechanics analysis based on differences within the plurality of three-dimensional models.

4. The computer-implemented method of claim 1, wherein facilitating treatment of the vascular structures using the result of the mechanics analysis includes recommending a treatment;
wherein recommending the treatment includes recommending at least one of a procedure type, a location, a stent length, a balloon size, a guidewire stiffness grade, a balloon inflation pressure, IVL pulse count and cycle parameters, or atherectomy burr sizing.

5. The computer-implemented method of claim 1, wherein facilitating treatment of the vascular structures using the results of the mechanics analysis includes determining optimal imaging coordinates, and wherein the method further comprises:
transmitting commands to an imaging system to cause the imaging system to collect images from the optimal imaging coordinates.

6. The computer-implemented method of claim 1, wherein generating the at least one three-dimensional model of the vascular structures using the classified and segmented plurality of medical images includes:
detecting common features in the medical images;
registering the medical images in a three-dimensional volume based on the common features; and
creating a three-dimensional mesh based on the registered medical images.

7. The computer-implemented method of claim 6, further comprising warping the three-dimensional mesh based on a predefined vascular structure template.

8. The computer-implemented method of claim 1, wherein conducting the mechanics analysis using the three-dimensional model includes:
conducting boundary and diameter quantification using the three-dimensional model;
conducting directional and curvature analysis using the three-dimensional model; and
determining one or more metrics using the three-dimensional model.

9. The computer-implemented method of claim 8, wherein the one or more metrics include at least one of:
an anatomical resistance characterization;
a fluid flow resistance characterization;
an obstruction classification;
a contrast washout calculation;
contrast intensity indexing;
a calcification identification; or
a tissue stiffness calculation.

10. The computer-implemented method of claim 1, wherein receiving the plurality of medical images includes at least one of:
receiving the plurality of medical images from an imaging system in real time from an x-ray angiography system, a CT scanning system, or an MRI system; or
retrieving the plurality of medical images from an image data store.

11. A non-transitory computer-readable medium having computer-executable instructions stored thereon that, in response to execution by one or more processors of a computing system, cause the computing system to perform actions for facilitating treatment of vascular structures of a subject, the actions comprising:
receiving, by the computing system, a plurality of medical images;
classifying, by the computing system, the medical images of the plurality of medical images by views, angles, and points in a cardiac cycle;
segmenting, by the computing system, the medical images of the plurality of medical images to detect vascular structures;
generating, by the computing system, at least one three-dimensional model of the vascular structures using the classified and segmented plurality of medical images;
conducting, by the computing system, a mechanics analysis using the at least one three-dimensional model; and
facilitating, by the computing system, treatment of the vascular structures using a result of the mechanics analysis.

12. The non-transitory computer-readable medium of claim 11, wherein generating the at least one three-dimensional model of the vascular structures using the classified and segmented plurality of medical images includes:
generating a plurality of three-dimensional models of the vascular structures, wherein each three-dimensional model is at a different point in the cardiac cycle.

13. The non-transitory computer-readable medium of claim 12, wherein conducting the mechanics analysis using the at least one three-dimensional model includes:
conducting, by the computing system, the mechanics analysis based on differences within the plurality of three-dimensional models.

14. The non-transitory computer-readable medium of claim 11, wherein facilitating treatment of the vascular structures using the result of the mechanics analysis includes recommending a treatment;
wherein recommending the treatment includes recommending at least one of a procedure type, a location, a stent length, a balloon size, a guidewire stiffness grade, a balloon inflation pressure, IVL pulse count and cycle parameters, or atherectomy burr sizing.

15. The non-transitory computer-readable medium of claim 11, wherein facilitating treatment of the vascular structures using the results of the mechanics analysis includes determining optimal imaging coordinates, and wherein the actions further comprise:
transmitting commands to an imaging system to cause the imaging system to collect images from the optimal imaging coordinates.

16. The non-transitory computer-readable medium of claim 11, wherein generating the at least one three-dimensional model of the vascular structures using the classified and segmented plurality of medical images includes:
detecting common features in the medical images;
registering the medical images in a three-dimensional volume based on the common features; and
creating a three-dimensional mesh based on the registered medical images.

17. The non-transitory computer-readable medium of claim 16, wherein the actions further comprise warping the three-dimensional mesh based on a predefined vascular structure template.

18. The non-transitory computer-readable medium of claim 11, wherein conducting the mechanics analysis using the three-dimensional model includes:
conducting boundary and diameter quantification using the three-dimensional model;
conducting directional and curvature analysis using the three-dimensional model; and
determining one or more metrics using the three-dimensional model.

19. The non-transitory computer-readable medium of claim 18, wherein the one or more metrics include at least one of:
an anatomical resistance characterization;
a fluid flow resistance characterization;
an obstruction classification;
a contrast washout calculation;
contrast intensity indexing;
a calcification identification; or
a tissue stiffness calculation.

20. A system, comprising:
an imaging system configured to generate medical images; and
an image analysis system comprising one or more processors and a non-transitory computer-readable medium having computer-executable instructions stored thereon that, in response to execution by the one or more processors, cause the image analysis system to perform actions comprising:
receiving, by the computing system, a plurality of medical images generated by the imaging system;
classifying, by the computing system, the medical images of the plurality of medical images by views, angles, and points in a cardiac cycle;
segmenting, by the computing system, the medical images of the plurality of medical images to detect vascular structures;
generating, by the computing system, at least one three-dimensional model of the vascular structures using the classified and segmented plurality of medical images;
conducting, by the computing system, a mechanics analysis using the at least one three-dimensional model; and
facilitating, by the computing system, treatment of the vascular structures using a result of the mechanics analysis.
